(19) United States
(12) Patent Application Publication
(10) Pub. No.: US 2004/0167459 A1
(43) Pub. Date: Aug. 26, 2004

(54) Methods and systems for controlling and/or increasing iontophoretic flux

(76) Inventors: William I. Higuchi, Salt Lake City, UT (US); David J. Miller, Bountiful, UT (US); S. Kevin Li, Salt Lake City, UT (US)

Correspondence Address: REED & EBERLE LLP, 800 MENLO AVENUE, SUITE 210, MENLO PARK, CA, 94025, US

(21) Appl. No.: 10/371,148

(22) Filed: Feb. 21, 2003

(51) Int. Cl. A61N 1/30

(52) U.S. Cl. 604/20

(57) ABSTRACT

An iontophoretic method is provided to selectively transport a compound of interest through a localized region of an individual's body tissue that exhibits a low electrical resistance and/or a high permeability. The method involves placing a permselective material, typically having a resistance comparable or higher than the resistance of the localized region, in ion-conducting relation to the localized region. An electrical current is then applied through the permselective material to the localized region, thereby transporting the compound of interest iontophoretically through the localized region. When the permselective material is capable of hindering iontophoretic transport of a competing ion, the transference efficiency of the compound of interest is increased during iontophoresis. As a result, the compound of interest is delivered into or extracted from the localized region at an enhanced rate. As transport efficiencies approach unity, absolute predictivity of transport also becomes possible. Also provided are electrode assemblies and iontophoretic systems capable of carrying out the iontophoretic method.

### TECHNICAL FIELD

This invention relates generally to methods and systems for selectively transporting a compound of interest through a localized region of an individual's body tissue, wherein the localized region exhibits an electrical resistance less than 100 kΩ-cm². More particularly, the invention involves the use of a permselective material that is capable of hindering iontophoretic transport of a competing ion that reduces transference efficiency of the compound of interest.

### BACKGROUND

Non-invasive drug delivery continues to be the focus of significant developmental efforts. Iontophoresis is a well-known noninvasive technique that may be used to deliver a compound of interest to, or to extract a compound of interest from, a body tissue of a patient. In practice, two iontophoretic electrodes are placed on a body tissue, typically the skin or mucosa, in order to complete an electrical circuit. At least one of the electrodes is considered to be an active iontophoretic electrode, while the other may be considered as a return, inactive, or indifferent electrode. The compound of interest is transported at the active electrode across the tissue as a permeant when a current is applied to the electrodes through the tissue. Compound transport may occur as a result of a direct electrical field effect (e.g., electrophoresis), an indirect electrical field effect (e.g., electroosmosis), electrically induced pore or transport pathway formation (electroporation), or a combination of any of the foregoing.

A majority of the known iontophoretic methods employ a constant direct current (DC) iontophoretic signal, and suffer from a number of shortcomings as a consequence. As a whole, the overarching problem associated with DC iontophoretic delivery is its high degree of variability. Contrary to simplified iontophoretic theory, a constant driving force provided by a direct current will not generally produce a constant, unwavering permeant flux. Constant DC typically causes the electrical resistance of the tissue to change as a result of variations in tissue porosity, pore surface charge density, and effective pore size over the course of treatment. As a result, the amount of compound transported across a tissue varies with time and cannot be controlled, monitored, or predicted effectively.

In addition, iontophoretic techniques that employ a constant DC signal can result in the formation of unwanted byproducts. For example, the application of a constant direct current to a tissue can result in water hydrolysis at the treatment site, causing protons to accumulate at the anode and hydroxide ions to accumulate at the cathode. The resulting shift in pH at the electrodes may cause tissue irritation and/or damage and may cause degradation of the compound of interest. In extreme cases, this resulting electrolysis causes gas formation at the interface between the active electrode and the tissue in contact with it. As a consequence, interfacial electrical resistance may be altered. In addition, the highly mobile hydrogen and hydroxide ion byproducts of water hydrolysis compete against the permeant for the electrical current, thereby decreasing permeant transport efficiencies.

Various techniques have been proposed to counter the deleterious effects of the unwanted byproducts. To avoid hydrolysis, a sacrificial electrode may sometimes be used during iontophoresis, wherein the electrode is oxidized or reduced at a lower potential than water. For example, a Ag/AgCl sacrificial electrode system may be used. However, Ag⁺ and Cl⁻ ions are small, highly mobile ions that may compete with a compound of interest for the iontophoretic current, thereby reducing transference efficiency of the compound of interest. Further, if allowed to proceed into the skin unimpeded, the Ag⁺ ions will stain the skin dark brown to black for weeks.

Deleterious effects of unwanted iontophoretic byproducts may sometimes be reduced through the use of an ion exchange medium that has either the same or the opposite charge as the drug to be delivered. See, e.g., U.S. Pat. Nos. 5,362,308, 5,250,022, 6,289,242, 6,049,733, 5,871,460, 5,084,008, 6,254,883, 4,722,726, 4,585,652, 5,232,438, 5,322,502, 5,169,382, 5,080,646, 5,169,383, 6,394,994, 4,731,049, 5,620,580, 6,330,471, 5,853,383, 6,071,508, 5,006,108, 5,871,461, 5,788,666, 5,840,056, 5,941,843, 5,993,435, 5,857,992, 5,503,632, 5,496,266, 4,927,408, 5,647,844, 4,915,685, 5,882,677, 6,394,994, 6,289,242, 6,049,733, 5,084,008, 5,057,072, 5,871,460, 5,993,435, 5,857,992, 5,496,266, 5,647,844, 5,853,383, 6,071,508, and 5,169,383. The medium may be used, for example, to scavenge, bind, chelate, or neutralize the byproducts. Often, such an ion exchange medium is provided in the form of a permselective membrane. For example, U.S. Pat. No. 5,395,310 to Untereker et al. describes an iontophoresis electrode for use on the skin of a patient. The electrode is comprised of: a conductive element, current distributing member; a drug reservoir electrically coupled to the current distributing member; and a charge selective material. In operation, the current distributing member is coupled to a source of direct electrical current, and the material is placed in contact with the patient's body surface. The material is interposed between the reservoir and the body surface. The material selects for ions having the same charge as the drug when ionized. Thus, when the electrode is used to deliver a positively charged drug into the patient's body tissue, the charge selective material allows passage of the drug therethrough, but prevents the passage of negatively charged ions, such as chloride ions, from migrating from the body and into the electrode.

This technology, however, suffers from a number of serious drawbacks. It is well known that iontophoresis can cause irritation, sensitization, and pain at the application site. The degree of irritation, sensitization, and/or pain is directly proportional to the applied current or voltage. In direct current transdermal iontophoretic systems, such as that described in U.S. Pat. No. 5,395,310 to Untereker et al., 0.5 mA/cm² is recognized as the maximum tolerable current density. With such current densities, skin will not typically become sufficiently permeable to allow for transdermal drug delivery or analyte extraction.

Iontophoretic methods that use alternating current (AC) signals, with or without a DC offset, have exhibited improved performance for both compound delivery and extraction. The premise of AC constant conductance iontophoresis is that molecular transport flux across a tissue is directly proportional to the tissue's conductivity and inversely related to the tissue's resistivity. It has been found that, at constant current levels, the molecular transport though a membrane is related to the conductance of the membrane. AC iontophoretic methods are described in U.S. Pat. No. 6,512,950 to Li et al., which corresponds to International Patent Publication No. WO 01/60449. AC iontophoretic methods are also described in U.S. Pat. No. 6,496,728 to Li et al., which corresponds to International Patent Publication No. WO 01/60448.

In order to reduce the energy requirements needed to effect iontophoretic transport, it has been discovered that application of a barrier-modifying substance (also referred to herein as a “barrier-modifying agent” or “barrier modifier”) to the body tissue, either prior to or during AC iontophoresis, lowers the potential voltage difference needed to achieve electroporation. As discussed in U.S. patent application Ser. No. 10/014,741, entitled “Method of Increasing the Battery Life of an Alternating Current Iontophoresis Device Using a Barrier-Modifying Agent,” filed on Dec. 10, 2001, the use of such barrier modifiers makes it possible to maintain the rate at which a compound of interest can be transported through a body tissue at lower electrical voltage levels. This reduction in applied voltage ultimately results in reduced battery requirement, reduced treatment duration, decreased treatment cost, and increased patient comfort.

The major problem associated with most iontophoretic systems is the high degree of flux variability during iontophoresis. Typically, flux variability is caused by variations in the effective electromobility of the current-carrying ionic species, as well as variations in the concentration of the ionic species in a patient's tissue under normal or disease states. In addition, variations in iontophoretic flux are typically exhibited from site to site and patient to patient. Given that the transference efficiency of a drug for any particular iontophoretic current is typically less than about 10%, roughly 90% or more of the iontophoretic current is typically used to transport competing ions. Thus, varying the total current applied for iontophoretic drug delivery is not fully effective for controlling or predicting the actual amount of drug delivered into a body or the target organ.

Thus, there is a need in the art to overcome the above-described drawbacks by increasing and/or controlling permeant flux during iontophoresis.

### SUMMARY OF THE INVENTION

One aspect of the invention relates to an iontophoretic method for selectively transporting a compound of interest through a localized region of an individual's body tissue that exhibits a low electrical resistance and/or a high permeability. The method involves placing a permselective material in ion-conducting relation to the localized region. An electrical current, AC, DC, or AC with superimposed DC, is then applied through the permselective material to the localized region, thereby transporting the compound of interest iontophoretically through the localized region. When the permselective material is capable of hindering iontophoretic transport of a competing ion, the transference efficiency of the compound of interest is increased during iontophoresis. As a result, the invention allows a compound of interest to be delivered into or extracted from the localized region more efficiently than previously known iontophoretic methods and devices.

The method is particularly suited for iontophoretic transport of a compound of interest through a localized region of tissue that exhibits a low electrical resistance that (e.g., less than 100 kΩ) which corresponds to a high tissue permeability. Thus, the inventive method is particularly suited for: mucosal tissue, e.g., oral or buccal tissue; and ocular tissue, e.g., scleral or conjunctival tissue. In addition, the localized region may be permeabilized so as to exhibit a higher permeability. Thus, the inventive method may be used for iontophoretic transport of a compound of interest through permeabilized mucosal or skin tissue.

Typically, the permselective material has an electrical resistance greater than the electrical resistance of the localized region, and can be provided in the form of a membrane. In addition, the permselective material is typically capable of hindering iontophoretic transport of a competing counter-ion that possesses a charge opposite to the charge of the ionized compound of interest.

In another aspect, the invention provides an iontophoretic electrode assembly for selectively transporting a compound of interest through a localized region of an individual's body tissue. The electrode assembly may be used to carry out the inventive method, and is comprised of an electrode adapted for electrical connection to a current source and a permselective material in ion-conducting relation to the electrode and having a surface adapted for contact with the localized region. The permselective material is capable of hindering iontophoretic transport of a competing ion that reduces transference efficiency of the compound of interest when the material is in contact with the localized region. The permselective material has an electrical resistance greater than the electrical resistance of the localized region, and the localized region exhibits an electrical resistance that corresponds to a tissue permeability that meets or exceeds the permeability of the individual's unpermeabilized skin tissue. Typically, the permselective material is provided as a membrane having a surface sized and/or shaped for direct contact with the localized region.

When the electrode assembly is used to deliver a compound of interest, e.g., a pharmacologically active agent into the localized region, the compound of interest may be contained within the permselective material. In addition or in the alternative, the electrode assembly may further comprise a reservoir, optionally containing the compound of interest. Typically, the reservoir is interposed between the electrode and the permselective material. In addition, the electrode assembly may further comprise a means for isolating the reservoir so as to prevent a redox product from entering the reservoir. For example, the means for isolating the reservoir may be comprised of an agent that precipitates, neutralizes, repels and/or binds to the redox product so as to prevent the product from entering the reservoir.

Optionally, the electrode assembly may further comprise a means for permeabilizing the localized region. The means for permeabilizing the localized region may comprise a chemical permeation enhancer, electroporation current, ultrasound, photons, a piercing member, or combinations thereof. For example, a chemical permeation enhancer may be contained within an applicator that applies the chemical permeation enhancer to the tissue prior to iontophoresis. In addition or in the alternative, the chemical permeation enhancer may be provided in ion-conducting relation to, or contained in, the permselective material. Furthermore, the means for permeabilizing the localized region may comprise a permeabilizing current applicator spaced apart from the electrode.

In a further aspect, the invention relates to an iontophoretic system for selectively transporting a compound of interest through a localized region of an individual's body tissue. The system employs a permselective material capable of selectively hindering iontophoretic transport of a competing ion when the material is in contact with the localized region, wherein transport of the competing ion reduces transference efficiency of the compound of interest. Also provided are a first electrode adapted to be placed in ion-conducting relation through the permselective material to the localized region to allow iontophoretic transport of the compound therethrough, and a second electrode adapted to contact the individual's body and spaced apart from the first electrode. A current source is connected electrically to the first and second electrodes, for applying an electrical current to the localized region of body tissue to transport the compound of interest iontophoretically through the localized region. Furthermore, the system includes a means for permeabilizing the localized region such that the permselective material has an electrical resistance greater than the electrical resistance of the localized region after permeabilization by the means for permeabilizing the localized region.

In yet another aspect, an alternating current source is electrically connected to the first and second electrodes, for applying an alternating current to the localized region to transport the compound of interest. The alternating current, with or without superimposed DC offset, is capable of simultaneously permeabilizing the membrane and transporting the compound of interest.

### BRIEF DESCRIPTION OF THE DRAWINGS

FIG. 1 schematically depicts the experimental setup for the human epidermal membrane (HEM) iontophoretic experiments described herein.

FIG. 2 is a graph that plots flux versus current level with and without permselective membrane and shows that presence of the permselective membrane increases permeant flux by approximately three-fold.

### DETAILED DESCRIPTION OF THE INVENTION

### Definitions and Nomenclature

Before describing the present invention in detail, it is to be understood that this invention is not limited to any specific drug delivery system, reverse iontophoresis extraction system, iontophoretic electrode assembly structure, iontophoretic method, permselective material, carrier, or the like, as such may vary. The definitions that follow apply only to the terms as they are used herein and may not be applicable to the same terms as used elsewhere, for example in scientific literature or other patents or applications, including other applications by these inventors or assigned to common owners. The following description of the preferred embodiments and examples is provided by way of explanation and illustration only, and is not intended to be limiting. As such, the preferred embodiments and examples are not to be viewed as limiting the scope of the invention as defined by the claims. Additionally, when examples are given, they are intended to be exemplary only and not to be restrictive.

It must be noted that, as used in this specification and the appended claims, the singular forms “a”, “an,” and “the” include plural referents unless the context clearly dictates otherwise. Thus, for example, reference to “a permselective material” includes a mixture, composite, or laminate of two or more such materials, either similar or dissimilar in nature, as well as a single permselective material; reference to “a compound of interest” includes one or more compounds of interest; reference to “a competing ion” includes one or more competing ions; and the like.

In describing and claiming the present invention, the following terminology will be used in accordance with the definitions set out below.

As used herein, a “body tissue” refers to an aggregation of similar cells and/or cell components united in performance of a particular function. The tissue can be part of a living organism, a section excised from a living organism, or artificial. For example, the tissue may be comprised of a section, or the entirety, of an internal or external organ. Typically, however, the body tissue will be a body surface of an individual, i.e., skin, mucosal tissue (including the interior surface of body cavities that have a mucosal lining, such as buccal tissue), ocular tissue (e.g. conjunctiva, sclera, and cornea), etc. In addition, the individual is typically human. The invention, however, also finds utility on small mammals, birds, farm and other domesticated animals, as well as animals found in the wild and in zoological parks.

The term “competing ion” is a charged species that carries iontophoretic current so as to reduce transference efficiency of the compound of interest during iontophoresis. Typically, though not necessarily, the compound of interest and the competing ion are of opposite charges. In such instances, the competing ion is considered a “counter” ion. Alternatively, a competing ion may be a “co-ion,” an ion that is transported in the same direction as the compound of interest and has the same charge as the compound of interest.

The term “compound of interest” is used collectively to refer to “drugs” and “analytes,” and includes charged and uncharged species, ions, molecules, chemical compounds, and compositions. Typically, a “compound of interest” is a “permeant” that is iontophoretically transported through a localized region of an individual's body tissue. It should be noted, however, that a “permeant” is not necessarily a “compound of interest.”

Typically, the terms “drug,” “active agent,” and “pharmacologically active agent” are interchangeably used to refer a charged or uncharged compound suitable for administration to an individual to produce a beneficial biological effect, preferably a therapeutic effect in the treatment of a disease or abnormal physiological condition, although the effect may also be prophylactic in nature. The terms also encompass agents that are administered for nutritive or diagnostic purposes, e.g., nutrients, dietary supplements, and imaging agents. The terms also encompass pharmaceutically acceptable, pharmacologically active derivatives of those active agents specifically mentioned herein, including, but not limited to, salts, esters, amides, prodrugs, active metabolites, analogs, and the like.

In contrast, the term “analyte” is typically used to refer to a compound, molecule, or ion to be iontophoretically extracted from a localized region of a patient's body tissue. When particular types of analytes are mentioned, it is to be understood that salts, esters, amides, analogs, conjugates, metabolites, and other derivatives are included unless otherwise indicated.

The terms “current” and “electrical current,” when used to refer to the conductance of electricity by movement of charged particles, are not limited to “direct electrical current,” “direct current,” or “constant current.” The terms “current” or “electrical current” should also be interpreted to include “alternating current,” “alternating electrical current,” “alternating current with direct current offset,” “pulsed alternating current,” and “pulsed direct current.”

The term “electrode” is used herein to refer to any terminal that conducts an electric current into or away from a conducting medium. Thus, an iontophoretic electrode is an electrode that conducts an electric current into or away from tissue. When a pure DC signal, or AC signal with a DC signal offset, is used, the “anode” is the electrode that receives a more positive contribution of the signal, whereas the “cathode” is the electrode that receives a more negative contribution of the signal. When pure AC is used, there is no formal anode or cathode.

The terms “iontophoresis,” “iontophoretic,” and “iontophoretically” are used herein to refer to the transport of a compound or ion through a localized region of a body tissue by means of an applied electric field that results in or is accompanied by a motive force. The terms “iontophoresis” and “iontophoretic” are also meant to refer to mechanisms such as “reverse iontophoresis,” “reverse iontophoretic,” “electroosmosis,” and “iontohydrokinesis” or “iontohydrokinetic.” Thus, for example, “iontophoresis” may involve delivery of a compound of interest into or through a localized region of a body tissue and/or extraction of an analyte through or from the localized region by means of an applied electromotive force.

During iontophoresis, certain modifications or alterations of the localized region of the body tissue, for example changes in permeability, may occur, due to mechanisms such as the formation of transiently existing pores, also referred to as “electroporation.” Any electrically assisted transport of species enhanced by modifications or alterations to the body surface (e.g., formation of pores, transiently or permanently existing in the skin, and “electroporation”) is also included in the term “iontophoresis” as used herein. Further, these terms include the transport of one or more compounds by passive, Fickian driven diffusion, either concurrent with or subsequent to tissue electroporation by the electrical field. Thus, as used herein, the terms “iontophoresis” and “iontophoretic” further refer to the transport of a species by the application of an electric field, regardless of the mechanisms.

A “localized region” of a tissue refers to the area or section of a body tissue through which a compound of interest is transported. Thus, a localized region of a body surface refers to an area of skin, mucosal, ocular, or other tissue through which an active agent is delivered or an analyte is extracted.

The terms “optional” and “optionally” mean that the subsequently described circumstance may or may not occur, so that the description includes instances where the circumstance occurs and instances where it does not.

The terms “permeabilize,” “permeabilized,” and “permeabilizing” refer to an increase in the ability of a material to allow a compound of interest to be permeated or passed through. Thus, any unpermeabilized tissue will exhibit a lower degree of permeability to a compound of interest than the same tissue after it is permeabilized.

The term “permselective,” as used in “permselective material,” refers to a material that is more permeable to a compound of interest than to a potentially competing ion. The material is thus capable of hindering iontophoretic transport of an ion that may competitively reduce the transference efficiency of a compound of interest when the material is in contact with the localized region. The permselective material generally has an electrical resistance greater than that of the localized region.

The terms “resistance” and “electrical resistance” are interchangeably used herein in their ordinary sense and refer to the opposition of a body or substance to electrical current passing through it, resulting in a conversion of electrical energy into heat or another form of energy. While the term may be used to describe the opposition of a body or substance to a DC signal, the term “resistance” is also used to refer to its AC analogue, “impedance,” which is a measure of the total opposition to current flow in an alternating current circuit, made up of two components—Ohmic resistance and reactance. The term “conductance” is also used herein in its ordinary sense and refers to the capacity of a body or substance to conduct electricity, which is measured as the reciprocal of resistance. Thus, in some instances, the terms “resistance” and “electrical resistance” refer to the Ohmic resistance of electrical current passage across a membrane, i.e., the voltage drop divided by the current applied. Typically, when used in reference to biomembranes or the “localized region,” the term “resistance” is evaluated in view of the passage of electrical current in the membrane's physiological state (e.g., in 0.15 M balanced salt solution).

The term “transport,” as in the “transport” of a compound of interest through a localized region of a body tissue, refers to passage of the compound in either an inward or outward direction. That is, the compound may be delivered to an individual from an external source, or extracted from-the individual, as in analyte extraction.

The term “transference efficiency”, “transference number”, or “electrical transference” refers to the ratio of current carried by the compound of interest during iontophoresis to the total iontophoretic current applied. When the compound of interest carries all of the current applied, the transference efficiency is unity, or 100%.

In general, the invention stems from the discovery that exceptional control over iontophoretic flux accompanies the use of a permselective material to transport a compound of interest through highly permeable body tissue. The permselective material serves to hinder iontophoretic transport of a competing ion that reduces transference efficiency of the compound of interest. As the transport efficiency approaches unity, the variability in flux commonly observed during electrophoresis, electroosmosis, passive transport, Fickian diffusion, and the like is virtually eliminated and the extent of permeant transport can be predicted based solely on Faraday's law.

The inventive methods and devices described herein reduce possible adverse events of maltherapeutic drug concentrations due to drug delivery variability, reduce the need for the high iontophoretic current requirements resulting from low efficiency iontophoretic transport, and overcome other limitations of conventional iontophoresis. For example, the enhancement in iontophoretic efficiency provides for therapeutic drug delivery in shorter periods of time, using lower current densities and lower drug concentrations in the donor solution. As a result, patient comfort is increased, irritation potential is reduced, and operational costs are lowered. Utilizing the control and programmability associated with the present invention, iontophoresis becomes a relatively simple regimen and a convenient procedure for drug administration and/or analyte extraction, with enhanced patient compliance and improved therapeutic outcomes.

In order to fully elucidate the novelty and nonobviousness of the invention, the following generalized discussion relates to the theory and practice of iontophoresis in the context of drug delivery. In particular, the discussion points out the limitations and drawbacks associated with known iontophoretic technologies. One of ordinary skill in the art should be able to recognize that similar considerations are also applicable to iontophoretic analyte extraction practices.

In general, iontophoretic devices utilize at least two electrodes. First and second electrodes are each positioned so as to be in electrical contact with a localized region of a body tissue, e.g., eye, skin, or mucosal tissue. The first electrode is typically referred to as the “active” or “donor” electrode and contains the drug to be delivered into the body. The second electrode, typically referred to as the “counter” or “return” electrode, serves to close an electrical circuit through the body tissue. When the drug to be driven into the body is positively charged, the positive electrode (the anode) acts as the active electrode and the negative electrode (the cathode) serves as the counter electrode, thereby completing the circuit. Conversely, if the drug to be delivered is negatively charged, then the cathode is the active electrode and the anode is the counter electrode. The device is also equipped with a reservoir connected to one of the electrodes to provide a source of the drug to be delivered. An electrical circuit is formed by connection of these electrodes to a source of electrical energy, e.g., a battery, and to circuitry capable of controlling the amount of current passing through the device and localized region.

During iontophoretic drug delivery, the donor electrode is typically placed in direct contact with the localized region, and the return electrode is placed on the body tissue, apart from the donor electrode. The donor electrode may have any of a number of different constructions. Typically, the donor electrode has a compartment that houses the drug to be delivered. For example, the donor electrode may be a sustained-release drug delivery device comprising a matrix saturated with the drug, or a polymer containing the drug. The drug will be transported across the localized region into the body tissue with the assistance of an electric field according to the following equation:

$${J}_{i} = \frac{{t}_{i} \times {I}_{total}}{F \times {z}_{i}} \qquad ( 1 )$$

where

Jᵢ=iontophoretic flux of the drug i

Iₜₒₜₐₗ=total current applied

F=Faraday's constant

zᵢ=the charge of the drug i

tᵢ=transference efficiency of the drug.

Transference efficiency defines the fraction of the current carried by the drug and is generally defined as the ratio of the current carried by the drug to the total current carried by all ionic species in solution. Thus:

$${t}_{i} = \frac{{I}_{i}}{{I}_{total}} \qquad ( 2 )$$

and

$${t}_{i} = \frac{{z}_{i} {J}_{i}}{{\sum }_{j} {z}_{j} {J}_{j}} \qquad ( 3 )$$

where

Iᵢ=current carried by drug i

zⱼ=charge of competing ion j

Jⱼ=flux of competing ion j

The competing ions j in equation (3) represent both the ions migrating into the body from the donor electrode and the oppositely charged counter-ions migrating into the electrode from the body. The ions migrating from the donor electrode are ions of the same charge as the polarity of the electrode. They can originate from the electrode or be introduced by electrochemical reactions of the electrode during iontophoresis. The ions migrating from the body are usually endogenous ions having a charge opposite to the polarity of the electrode.

When electrophoresis is the dominant driving force of the drug through the localized region, the flux of a competing ion j is a function of the effective mobility of the competing ion in the localized region, the concentration of the competing ion in its respective system, and the valence or charge of the competing ion:

$$J = μ C \frac{ⅆ ψ}{ⅆ x} \qquad ( 4 )$$

where

μ=effective electromobility of a competing ion

C=concentration of the ionic species

dψ/dx=electric field.

To maximize iontophoretic flux of the drug, it is a normal practice to exclude from the donor compartment co-ions of the drug, i.e., ions that are transported in the same direction as the drug (also known as “background ions,” “background electrolytes,” or “excipient ions”). With this practice, with only the drug in the donor compartment, the ions generated by the electrochemical reactions at the electrode's surface, and the ions migrating outwardly from the body contribute to the current across the localized region. For anodic delivery of positively charged drugs, positive ions generated at the anode surface (e.g., Ag⁺ ions when a Ag anode is used) and chloride ions extracted from the body are the main ionic competitors to the drug's electrical transference. For cathodic delivery of negatively charged drugs, sodium ions extracted from the body and negative ions generated from the cathode surface (e.g., Cl⁻ ions when a AgCl cathode is used) are the main competitors to drug's electrical transference. Under normal conditions, the concentrations of the endogenous sodium and chloride ions can be substantially higher than the concentration of the drug in the donor electrode. Also, depending on the molecular size and charge of the drug to be delivered, the effective electromobilities of these small ions are often several-fold higher than the electromobility of the drug to be delivered.

When the drug is positively charged and there are no co-ions present in solution, the transference efficiency of the drug through a localized region of a body tissue can be expressed by:

$${t}_{i}^{lr} = \frac{{z}_{i} {μ}_{i}^{lr} {C}_{d}}{{z}_{Cl} {μ}_{Cl}^{lr} {C}_{Cl}^{body} + {z}_{i} {μ}_{i}^{lr} {C}_{d}} \qquad ( 5 )$$

where

C_{d}=drug concentration in the donor electrode compartment

C_{Cl}^{body}=Cl⁻ ion concentration in the tissue fluid underlying the localized region

subscript i refers to the drug i

subscript Cl refers to the Cl⁻ ion

superscript lr refers to the localized region

The low concentration and low mobility of the drug compared with the endogenous Na⁺ or Cl⁻ reduce the drug's electrical transference (equations 1 to 4) to low values. The high proportion of current carried by the endogenous Na⁺ or Cl⁻ and the ions introduced into the system from the redox reaction at the electrode surface greatly limit iontophoretic drug delivery, producing delivery with low efficiency. Typical iontophoretic efficiencies are on the order of less than 2-10%.

Accordingly, while iontophoresis can, in theory, have a significant advantage over other methods of drug administration, the flux variability that occurs with previously known iontophoretic methods commonly results in inaccurate dosing. Known iontophoretic technologies are incapable of precisely controlling the amount of drug delivered into a body tissue. Such limitations also render known iontophoretic technologies unsuitable for delivering drugs that require precise control over the delivery rate (e.g., low therapeutic index drugs).

In contrast, the present invention is capable of effecting iontophoretic transport of a compound of interest through a localized region of a body tissue with sufficient control over permeant flux so as to overcome the limitations associated with previously known iontophoretic devices. As discussed above, the invention is suited for effecting iontophoretic transport of a compound of interest through a localized region of a body tissue having an electrical resistance that corresponds to an intrinsic high permeability. In addition, permeability and electrical resistance of a body tissue are roughly inversely proportional to each other when solution ionic strength is constant. Since the invention is particularly suited for highly permeable tissue, it is preferred that the tissue exhibits a low electrical resistance. Typically, the localized region exhibits an electrical resistance no greater than about 10% of the electrical resistance of unpermeabilized tissue or possesses an inherently low resistance before permeabilization. Preferably, the electrical resistance of the localized region is no greater than about 1% of the electrical resistance of unpermeabilized tissue for inherently low permeability tissues, such as skin. For all tissues, both with inherently high and low permeability, it is preferred that the electrical resistance of the localized region be less than 10 kΩ-cm², more preferably less than 5 kΩ-cm2, and optimally less than 2 kΩ-cm².

The invention also provides an iontophoretic electrode assembly for selectively transporting a compound of interest through a localized region of an individual's body tissue, wherein the localized region exhibits an electrical resistance that corresponds to a tissue permeability that substantially exceeds the permeability of the individual's unpermeabilized skin tissue. The electrode assembly is comprised of an electrode adapted for electrical connection to a current source, and a permselective material in ion-conducting relation to the electrode and having a surface adapted for contact with the localized region. The permselective material has an electrical resistance greater than or comparable to the electrical resistance of the localized region and is capable of hindering iontophoretic transport of a competing ion that reduces transference efficiency of the compound of interest when the material is in contact with the localized region. The inventive electrode assembly may be employed to practice the inventive method. However, the inventive method may also be practiced using other electrode means.

An important factor in the practice of the invention relates to the permeability of the tissue through which the compound of interest is transported. As alluded to above, permeability of a tissue is dependent on the quality or quantity of transport pathways present in the tissue. In some instances, the pathways may be endogenous to the tissue. In such cases, the invention may involve the transport a compound of interest through an unpermeabilized tissue of sufficiently high permeability. For example, mucosal tissue is typically significantly more permeable than unmodified skin tissue. Thus, the invention may be used to transport a compound of interest through oral or buccal tissue, as well as other mucosal tissues such as nasal, esophageal, intestinal, vaginal, or rectal tissue. In addition, it is well known that ocular tissue is significantly more permeable than unpermeabilized skin tissue. Thus, the invention may be used to effect permeant transport through scleral and/or conjunctival tissue.

In the alternative, the invention may be used to carry out iontophoretic transport through any permeabilized tissue that exhibits a lower resistance than 100 kΩ-cm². Thus, exemplary permeabilized tissues suitable for use with the invention include skin, mucosal, and ocular tissue. In some instances, permeabilization may reduce the electrical resistance of the localized region by at least about 80%. Preferably, permeabilization will reduce the tissue's electrical resistance by at least about 90%. Optimally, permeabilization will reduce the tissue's electrical resistance by at least about 99%.

Thus, the inventive electrode assembly may further comprise a means for permeabilizing the localized region, wherein the permeabilizing means is used prior to or concurrent with the application of an iontophoretic current. In some instances, permeabilization may be achieved through use of a chemical permeation enhancer. Such permeation enhancers are comprised of a compound or composition that is effective to alter the inherent barrier of a body tissue so as to facilitate transport of a compound of interest therethrough. For example, with skin, the stratum corneum serves as a cutaneous barrier through which most applied compounds and compositions will not penetrate. A permeation enhancer, in this context, is a compound that alters the stratum corneum so as to facilitate the transdermal transport of an actively delivered agent or an extracted analyte. Cutaneous barrier modifiers generally disrupt the stratum corneum barrier function by inserting into or otherwise disrupting the lipid bilayer structure in the intercellular regions within the stratum corneum, by inducing hydration and/or swelling of the lipid bilayer, by denaturing epidermal keratin, and/or by facilitating solubilization of the compound to be transported. In some instances, barrier-modifying agents that serve to enhance the barrier properties of a tissue may be used in conjunction with permeation enhancers to control the degree to which a tissue is permeabilized. Thus, a permeabilizing means may comprise a chemical permeation enhancer and an optional applicator that applies the chemical permeation enhancer. In some instances, the chemical permeation enhancer is provided in ion-conducting relation to the permselective material, e.g., within the permselective material.

In addition, tissue may be permeabilized through the appropriate application of electroporation current, ultrasound, photons, a piercing member, and combinations thereof. In addition or in the alternative, the means for permeabilizing the localized region comprises a permeabilizing current applicator spaced apart from the electrode. In such a case, the permselective material may be interposed between the electrode and the permeabilizing current applicator.

For full exploitation of the advantages associated with the invention, particularly in the context of drug delivery, the localized region should have a relatively low electrical resistance compared with the electrical resistance of the permselective membrane. Thus, the permselective material will typically have an electrical resistance greater than or comparable to the electrical resistance of the localized region. The electrical resistance of the permselective material is preferably at least two times the electrical resistance of the localized region, more preferably at least five times the electrical resistance of the localized region, and still more preferably at least ten times the electrical resistance of the localized region. Under these conditions, it is the resistance of the permselective material that effectively controls the iontophoretic current rather than the resistance of the localized region. Accordingly, by using a permselective material having a sufficiently high resistance compared to the resistance of the localized region, iontophoretic transport of a compound of interest may be controlled.

In the specific context of drug delivery, for example, the concentration of the drug at the permselective membrane/body tissue interface is typically controlled by the transference efficiency of the drug through the permselective membrane, and the counter-ion (e.g., Cl⁻) concentration in the membrane/body surface interface required to maintain electroneutrality. The transference efficiency of drug transport across the permselective membrane can be expressed by:

$${t}_{i}^{pm} = \frac{{z}_{i} {μ}_{i}^{pm} {C}_{i}^{d}}{{z}_{Cl} {μ}_{Cl}^{pm} {C}_{Cl}^{int} + {z}_{i} {μ}_{i}^{pm} {C}_{i}^{d}} \qquad ( 6 )$$

where

Cᵢ^{d}=drug concentration in the donor compartment of the electrode

C_{Cl}^{int}=chloride ion (counter ion) concentration at the membrane/body surface interface

superscript pm refers to the permselective membrane.

With this invention, the transference efficiency of the system will be controlled by the transference efficiency of the permselective material. When the transference efficiency of the drug across a permselective material is close to unity, the transference efficiency of the drug across the combined permselective material and body surface system (equation 6) can be considerably higher than that without the permselective material (equation 5). Optimally, the transference efficiency of the drug across the combined permselective membrane and body surface system also is close to unity, which can be 10 to 50 times more efficient than the transference efficiency calculated with equation 5 without the permselective membrane.

The electrical transference efficiency of the drug will ideally be unity. That is, the drug will be the only ion in the system carrying the electrical current. In such a system, absent passive and electroosmotic contribution to delivery, the amount of drug transported across the body surface can be accurately predicted by Faraday's law:

$$M = \frac{I \times time}{\vert z \vert \times F} \qquad ( 7 )$$

where

M=number of moles of drug transported

I=current flow

z=valence of drug ion

F=Faraday's constant (approximately 96,500 Coulomb/mole)

Any permselective material capable of hindering iontophoretic transport of a competing ion during iontophoretic transport of the compound of interest may be used in conjunction with the invention. Typically, a permselective material is capable of hindering iontophoretic transport of a competing counter-ion that possesses a charge opposite to the charge of the compound of interest when ionized. The competing ion may be positively or negatively charged. In addition, the material must be capable of being placed in ion-conducting relation to the localized region, and is preferably capable of establishing direct conformal contact with the localized region. While both organic and inorganic materials (e.g., certain ion-conducting ceramics) having permselective properties are known in the art, organic materials are preferred.

The permselective material may be provided in any of a number of forms. For example, the material may be provided in a fully liquid, partially liquid, gelled, partially solid, or fully solid state. For ease in handling, however, it is preferred that the permselective material be provided as a membrane. In some instances, such membranes are freestanding. Alternatively, the permselective material may be supported by a support structure such as an additional membrane having sufficient porosity and chemical inertness so as to avoid interfering with the performance of the permselective material, yet having sufficient mechanical integrity for ease in handling. In some instances, the permselective material may comprise regions capable of transferring an electrical current interdispersed within an insulating regions. Other forms of permselective materials may be employed as well. Optimally, the material is provided in the form of a membrane having a surface sized and/or shaped for direct contact with the localized region.

In some instances, the permselective material may be comprised of a polyelectrolyte. Although a polyelectrolyte may be a single molecule or an aggregate of molecules, such as a micelle or liposome, polyelectrolytes are more typically polymers having ions or ionizable groups. Such polyelectrolytes may be selected so as to have a molecular weight of about 200 Da or greater, e.g., in the range of 200 Da to 1000 Da, or to have a molecular weight of about 1000 Da or greater, e.g., in the range of 1000 Da to 10,000 Da. Such molecular weight ranges typically ensure that the polyelectrolyte will have a size sufficient to hinder its entrance into the transport pathways of the localized region, and that it is transported through body tissue very little, if at all, even under the influence of an electrical current. The polyelectrolyte may be cationic, anionic, nonionic, or amphoteric. In some instances, a plurality of polyelectrolytes of the same or different type may be employed. If the polyelectrolyte is particulate, i.e., comprised of a plurality of molecular aggregates, the particles can be porous or nonporous, and may be, for example, macromolecular structures such as micelles (cationic or anionic) or liposomes (cationic or anionic). Polyelectrolytes may be in solution form or present in a suspension, dispersion, or colloidal system.

Preferably, the polyelectrolyte is a compound having at least one ionic group. Exemplary cationic polyelectrolytes contain quaternary ammonium; primary, secondary, or tertiary amines charged at reservoir solution pH; heterocyclic compounds charged at reservoir solution pH; sulfonium; or phosphonium groups. Anionic polyelectrolytes typically contain one or more carboxylate, sulfonate, or phosphate groups. In addition, polyelectrolytes having characteristics of more than one of these categories may also be used in the methods of the invention. For example, partial hydrolysis of a compound such as polyacrylamide produces an amphoteric polyelectrolyte that has both amide (nonionic) and carboxylic acid (anionic) groups. Accordingly, the polyelectrolyte can comprise one or more ionic groups selected from the group consisting of quaternary ammonium, sulfonium, phosphonium, carboxylates, sulfonates, and phosphates. Exemplary backbone structures for such polyelectrolyte compounds include, by way of illustration and not limitation, acrylamides, addition polymers (e.g., polystyrenes), oligosaccharides and polysaccharides (e.g., agaroses, dextrans, celulloses), polyamines and polycarboxylic acid salts, polyethylenes, polyimines, polystyrenes, and mixtures thereof.

In addition, there are numerous other materials that are suitable for use as polyelectrolytes, either as is or by modification to include ionic groups. These include the following: heparin and heparin derivatives; liposomes, both anionic and cationic; micelles, both anionic and cationic; polyamines, such as polyvinylpyridine; polyethylenes, including chlorosulfonated polyethylene, poly(4-t-butylphenol-co-ethylene oxide-co-formaldehyde) phosphate, polyethyleneaminosteramide ethyl sulfate, poly(ethylene-co-isobutyl acrylate-co-methacrylate) potassium, poly(ethylene-co-isobutyl acrylate-co-methacrylate) sodium, poly(ethylene-co-isobutyl acrylate-co-methacrylate) sodium zinc, poly (ethylene-co-isobutyl acrylate-co-methacrylate) zinc, poly(ethylene-co-methacrylic acid-co-vinyl acetate) potassium, polyethyleneimine, and poly(ethylene oxide-co-formaldehyde-co-4-nonylphenol) phosphate; polysaccharides, including cross-linked polysaccharides such as agaroses, celluloses [e.g., benzoylated naphthoylated diethylaminoethyl (DEAE) cellulose, benzyl DEAE cellulose, triethylaminoethyl (TEAE) cellulose, carboxymethylcellulose, cellulose phosphate, DEAE cellulose, epichlorohydrin triethanolamine cellulose, oxycellulose, sulfoxyethyl cellulose, and QAE cellulose], starch, and the like; and mixtures thereof.

Notably, polyelectrolytes may be provided in the form of an ion-exchange resin. These ion exchange resins are sold under numerous tradenames such as Amberlite® and Amberjet® (both Rohm & Haas Company), Dowex® (Dow Chemical Co), Diaion® (Mitsubishi Kasei Corporation), Duolite® (Duolite International Inc.), Trisacryl® (Sepracor S.A. Corp.), and Toyopearl® (Toyo Soda Manufacturing Co., Ltd.). Additional information relating to polyelectrolytes and ion exchange resins can be found in U.S. patent application Ser. No. 10/226,622 for “Method for Stabilizing Flux and Decreasing Lag-Time During Iontophoresis,” filed Aug. 21, 2002, inventors Higuchi, Miller, Li, and Hastings.

The invention may be used both for permeant delivery and extraction, particularly in the context of medical treatment. When so employed for permeant extraction, the invention may be used, for example, to extract a substance through a body tissue for the purpose of quantitative or qualitative analysis. When so employed for permeant delivery, the invention may be used, as examples, for reduction in severity and/or frequency of symptoms, elimination of symptoms and/or underlying cause, prevention of the occurrence of symptoms and/or their underlying cause, or improvement or remediation of damage. When drug delivery is desired, the invention may be used to deliver a wide range of pharmacologically active agents. The methods can generally be utilized to deliver any chemical material or compound that induces a desired pharmacological, physiological effect, and that can be iontophoretically transported across tissue. In general, pharmacologically active agents that will be iontophoretically administered using the present method will be therapeutically effective, prophylactically effective, or cosmeceutically effective, and can be in any suitable form such as pharmaceutically acceptable, pharmacologically active derivatives and analogs of those active agents specifically mentioned herein, including, but not limited to, salts, esters, amides, prodrugs, active metabolites, inclusion complexes, analogs, and the like.

In some embodiments, two or more pharmacologically active agents are administered in combination, and are typically administered simultaneously. Further, a pharmacologically active agent can be combined with various agents that enhance certain aspects of transport. For instance, a first active agent can be combined with a second active agent that improves blood circulation, to enhance the rate of delivery of the therapeutic agent throughout a patient's body. Conversely, a first active agent can be combined with a second active agent that constricts local blood flow, to limit the diffusion of the compound to the general circulation and limit the first active agent's activity to the localized region of delivery. Other methods utilize one or more excipients that act to control the level of transport that occurs during the procedure.

The active agent will generally be delivered as a component of a pharmaceutical formulation suitable for topical, transdermal, transocular, and/or transmucosal administration, and will contain at least one pharmaceutically acceptable vehicle. Examples of vehicles typically used in such formulations are distilled water, buffered water, physiological saline, PBS, Ringer's solution, dextrose solution, and Hank's solution. In addition, the formulation can include other carriers, adjuvants, and/or non-toxic, non-therapeutic, nonimmunogenic stabilizers, excipients, and the like. The formulation may also include additional substances to approximate physiological conditions, such as pH adjusting and buffering agents, tonicity adjusting agents, wetting agents, detergents, and the like. Further guidance regarding formulations that are suitable for various types of administration can be found in Remington: The Science and Practice of Pharmacy 20^{th} edition (2000).

The pharmacologically active agent delivered using the present methods is administered in an amount effective for prophylactic and/or therapeutic purposes. An effective therapeutic amount is an amount sufficient to remedy a disease state or symptoms, or otherwise prevent, hinder, retard, or reverse the progression of a disease or any undesirable symptoms. An effective prophylactic amount is an amount sufficient to prevent, hinder, or retard a disease or any undesirable symptoms. The effective amount of any particular active agent will depend upon a number of factors known to those of skill in the art, including, for example, the potency and potential toxicity of the agent, the stability of the agent in the body, and the age and weight of the patient.

The active agents can also be compounds that are not delivered for a therapeutic or prophylactic purpose, but that are otherwise physiologically or medically useful. Such compounds include, by way of example, nutrients and imaging agents.

When transdermal drug delivery is desired, the pharmacologically active agent can be selected from the group consisting of β-agonists; analeptic agents; analgesic agents; anesthetic agents; anti-angiogenic agents; anti-arthritic agents; anti-asthmatic agents; antiangiogenic agents; antibiotics; anticancer agents; anticholinergic agents; anticoagulant agents; anticonvulsant agents; antidepressant agents; antidiabetic agents; antidiarrheal agents; anti-emetic agents; anti-epileptic agents; antihelminthic agents; antihistamines; antihyperlipidemic agents; antihypertensive agents; anti-infective agents; anti-inflammatory agents; antimetabolites; antimigraine agents; antiparkinsonism drugs; antipruritic agents; antipsychotic agents; antipyretic agents; antispasmodic agents; antitubercular agents; anti-ulcer agents; antiviral agents; anxiolytic agents; appetite suppressants; attention deficit disorder and attention deficit hyperactivity disorder drugs; cardiovascular agents; central nervous system stimulants; cytotoxic drugs; diuretics; genetic materials; hormonolytics; hypnotics; hypoglycemic agents; immunosuppressive agents; muscle relaxants; narcotic antagonists; neuroprotective agents; nicotine; nutritional agents; parasympatbolytics; peptide drugs; psychostimulants; sedatives; steroids; smoking cessation agents; sympathomimetics; photoactive agents; tocolytic agents; tranquilizers; vasodilators; and active metabolites thereof.

The invention can also be useful for drug delivery into the eye to treat serious or benign eye diseases. When transcleral drug delivery is considered, the therapeutic compound can be selected from the group of steroids, antibacterials, antivirals, antifungals, antiprotozoals, antimetabolites, VEGF inhibitors, ICAM inhibitors, antibodies, protein kinase C inhibitors, chemotherapeutic agents, neuroprotective agents, nucleic acid derivatives, aptamers, proteins, enzymes, peptides, polypeptides.

When analyte extraction is desired, any substance that is in the system or body (e.g., circulatory system, tissue system) of an individual and that can be transported across an electroporated or other permeabilized tissue may be extracted: a substance from within the individual's body may thus be transported through the localized region of the body surface to the exterior of the body. In some instances, the extracted compound is endogenous to the body tissue. Such analytes may correlate with particular diseases or disease states, and thus can be used in their diagnosis or monitoring. Exemplary molecular entities that are markers of disease states include, by way of illustration and not limitation, glucose, galactose, lactic acid, pyruvic acid, and amino acids such as phenylalanine and tyrosine. For example, glucose is useful for monitoring diabetic patients, phenylalanine levels can be ascertained to monitor the treatment of phenylketonuria (a condition that is manifested by elevated blood phenylalanine levels), galactose levels can be ascertained for patients with galactosemia, and so forth.

In addition, the extracted compounds can be pharmacologically active agents that have been administered to the subject, metabolites of such active agents, substances of abuse, electrolytes, minerals, hormones, amino acids, peptides, metal ions, nucleic acids, genes, enzymes, toxic agents, or any metabolites, conjugates, prodrugs, analogs, or other derivatives (e.g., salts, esters, amides) of the aforementioned substances. In some instances, more than one substance may be monitored at a time. Some specific monitoring applications are described below. The substances can be charged (negatively or positively), uncharged, or electronically neutral (e.g., zwitterionic substances with an equal number of opposing charges). In one embodiment, at least two analytes are extracted concurrently.

For example, the invention finds particular utility when the analyte is a pharmacologically active agent whose level in the blood requires monitoring. Exemplary pharmacologically active agents include those agents that have been administered to the patient for therapeutic or prophylactic treatment, and metabolites thereof, and include, by way of illustration and not limitation, β-agonists; analeptic agents; analgesic agents; anesthetic agents; anti-angiogenic agents; anti-arthritic agents; anti-asthmatic agents; antibiotics such as aminoglycoside antibiotics; anticancer agents; anticholinergic agents; antiangiogenic agents; anticoagulant agents (e.g., heparin, low molecular weight heparin analogues, and warfarin sodium); anticoagulants; anticonvulsant agents; antidepressant agents; antidiabetic agents; antidiarrheal agents; anti-emetic agents; anti-epileptic agents; antihelminthic agents; antihistamines; antihyperlipidemic agents; antihypertensive agents; anti-infective agents; anti-inflammatory agents; antimetabolites; antimigraine agents; antiparkinsonism drugs; antipruritic agents; antipsychotic agents; antipyretic agents; antispasmodic agents; antitubercular agents; anti-ulcer agents; antiviral agents; anxiolytic agents; appetite suppressants; attention deficit disorder and attention deficit hyperactivity disorder drugs; cardiovascular agents, including calcium channel blockers, antianginal agents, central nervous system (“CNS”) agents, beta-blockers, and antiarrhythmic agents, for example, cardiac glycosides; central nervous system stimulants; cytotoxic drugs; diuretics; genetic materials; hormonolytics; hypnotics; hypoglycemic agents (e.g., glucagon and other carbohydrates such as glucose); immunosuppressive agents; muscle relaxants; narcotic antagonists; neuroprotective agents; nicotine; nutritional agents; parasympatholytics; peptide drugs; psychostimulants; sedatives; steroids; smoking cessation agents; sympathomimetics; photoactive agents for photodynamic therapy; tocolytic agents; tranquilizers; vasodilators; and active metabolites thereof. Additional analytes that can be extracted from humans are discussed in “Iontophoresis Devices for Drug Delivery,” by Praveen Tyle, Pharmaceutical Research, vol. 3, no. 6, pp. 318-326, as well as in U.S. patent application Ser. No. 10/226,622 for “Method for Stabilizing Flux and Decreasing Lag-Time During Iontophoresis,” filed Aug. 21, 2002, inventors Higuchi, Miller, Li, and Hastings.

In addition, the inventive electrode assembly may include a reservoir for containing the compound of interest, wherein the reservoir is in electrical contact with the electrode and the permselective material. Typically, the reservoir is interposed between the electrode and the permselective material. In addition, when the electrode assembly is provided for drug delivery, the drug may be contained within the reservoir.

For optimal use, the invention further includes a means of preventing redox products generated at the electrode surface from entering the drug-containing zone/chamber. Redox products competing with the drug ion in iontophoretic transport will reduce the efficiency of drug delivery. For example, if an inert electrode is used for iontophoresis, water is hydrolyzed in one of the following reactions:

H₂O+e⁻→½H₂+OH⁻ (cathode reaction)

H₂O→2H⁺+½O₂+2e⁻ (anode reaction)

The hydrolysis products in a non-sacrificial system will result in severe changes in the pH of the donor electrode. Further, if allowed to proceed unimpeded into the drug electrode and the patient's skin, the acidic or caustic products will effectively compete with the drug for the electrical current, thereby decreasing transport efficiency. Further, such drastic changes in pH will likely degrade the drug and will almost certainly be a source of major skin irritation or damage. Lastly, the OH⁻ and H⁺ generated at the cathode and anode, respectively, typically exhibit a higher electrophoretic mobility than the drug ion, and will therefore have a degradatory effect on the electrical transference efficiency.

When a sacrificial electrode system is used, the electrode system itself generates redox products that may compete with the compound of interest for iontophoretic current. For example, when a Ag/AgCl system is used, the following reactions occur:

Ag₍ₛ₎→Ag⁺+e⁻ (anode reaction)

AgClM₍ₛ₎+e⁻→Ag₍ₛ₎+Cl⁻ (cathode reaction)

The Ag⁺ and Cl⁻ are small, highly mobile ions that compete very effectively with the drug for the electrical current, thereby decreasing the drug's electrical transference. Further, if allowed to proceed into the skin unimpeded, the Ag⁺ ions will stain the skin dark brown to black for weeks.

Thus, the inventive electrode assembly may optimally comprise a means for isolating the reservoir so as to prevent a redox product from entering the reservoir. As discussed above, the redox species will typically be Ag⁺ or H⁺ at the anode and Cl⁻ or OH⁻ at the cathode. The means for isolating the reservoir may be comprised of an agent that precipitates, neutralizes, and/or binds to the redox product so as to prevent the product from entering the reservoir. In addition or in the alternative, the isolating means may be comprised of an additional permselective material. Such additional permselective materials typically select for ions of opposite charge to those of the electrochemically generated species. In other words, the charge of the membrane is typically the same as the electrochemically generated species. The additional permselective material or other isolating means prevent entry of a redox product by providing an opposing electrochemical gradient.

Optionally, the compound of interest may be contained within the permselective material. For example, the permselective material may be of the charge opposite to that of the drug ion when the drug is pre-loaded into the permselective material. In such a case, the permselective material may exclude ions migrating from the body surface into the permselective material and into the drug reservoir. In addition, the permselective material may also prevent the ions generated in the redox reactions at the electrode surface from entering the drug reservoir until most of the drug is unloaded. As a result, an additional means will not be necessary to prevent the redox products from entering the drug reservoir.

The invention also provides an iontophoretic system for selectively transporting a compound of interest through a localized region of an individual's body tissue. As discussed above, a permselective material is provided that is capable of selectively hindering iontophoretic transport of a counter-ion when the material is in contact with the localized region, wherein transport of the counter-ions reduces transference efficiency of the compound of interest. The system also includes a first electrode and a second electrode spaced apart from the first electrode. A current source is electrically connected to the first and second electrodes. Preferably, an alternating current source is provided. In some instances, the current source may produce an AC signal having a superimposed DC signal. Alternatively, the current source may generate DC only. Iontophoresis occurs when the first electrode is placed in ion-conducting relation through the permselective material to the localized region, the second electrode is placed in contact with the individual's body to complete an iontophoretic circuit, and the current source applies an electrical current to the localized region of body tissue. In some embodiments, the permselective material has an electrical resistance greater than the electrical resistance of the localized region.

In some instances, the inventive iontophoretic system will have more than two conductive elements for transmitting the electrical current that will drive the drug ion through the localized region. This allows greater control over the electric field generated. It is anticipated that the permselective membrane will contribute significantly to the overall resistance of the system. This contribution may preclude achieving a high degree of permeability enhancement (i.e., by electroporation of the localized region) if only one conductive element is used in a classical electrode configuration, with the conductive element on the side of the electrode distal to the body surface.

In a preferred embodiment, the invention provides an electrode assembly as described above that has a reservoir containing the compound of interest and an additional electrode, preferably porous. The iontophoretic electrode is adjacent to the reservoir on the distal side of the permselective material in the electrode assembly, and the additional electrode is placed between the permselective membrane and the localized region. The additional electrode may function as a permeabilizing current applicator and may serve to enhance permeability of the localized region through electroporation without producing an accompanying large potential drop across the permselective material. Also, such an additional electrode will provide for a more uniform electric field across the body surface, thus inducing a more uniform pattern of permeabilization of the body surface. In such a configuration, the iontophoretic electrode would provide a direct current driving force for drug transport from the drug-containing component of the electrode, through the permselective material, through the porous second electrode, and through the localized region of the body tissue permeabilized by the permeabilizing current applied by the additional electrode.

Although the invention has utility for a wide range of iontophoretic applications, the invention is particularly suited for drug delivery to ocular tissue to treat diseases of the eye, particularly oculopathies such as posterior and intermediate uveitis, HSV retinitis, age related macular degeneration, diabetic retinopathy, bacterial, fungal, or viral endophthalmitis, eye cancers, glioblastomas, glaucoma, and glaucomatous degradation of the optic nerve. A variety of delivery methods currently exist to treat these conditions. Exemplary drug delivery methods to the posterior ocular chamber currently include: direct injection into the vitreous, systemic administration with subsequent distribution into the eye through optic blood flow, injection into the areas surrounding the globe with subsequent passive diffusion through the sclera into the globe, and topical application to the cornea and/or sclera with subsequent passive diffusion or iontophoretic enhanced delivery into the globe's interior. Each delivery method, however, suffers from its own shortcomings.

Generally, it is difficult to deliver therapeutically effective concentrations of drug into the eye via systemic routes, because the eye is an immunoprivileged organ. The blood vessels supplying the eye have tight junctions between their endothelial cells, preventing the transfer of most non-endogenous compounds from the blood to the eye's interior. In effect, a blood-retinal barrier is erected to inhibit entry of most systemically circulating drugs into the eye itself, thereby protecting the interior of the eye in a manner similar to that afforded the central nervous system by the blood-brain barrier. In order to achieve therapeutic concentrations in the eye following systemic delivery, large quantities of the drug must be administered to overwhelm the barrier. The increased quantities of the drug in the systemic circulation, in turn, expose the entire body to the adverse effects of the drugs. Many such drugs exhibit whole-body toxicity at the high systemic concentrations required for ocular delivery.

For example, when a steroid is administered in large doses to a patient, such as for the treatment of uveitis, adverse effects induced upon the entire body can include fluid retention, electrolyte imbalance, immunosuppression, myopathy, cataract formation, behavior changes, bone demineralization, and others. Similarly, if large doses of a vascular endothelial growth factor (VEGF) antagonist are administered systemically, adverse effects could include delayed wound or injury healing and decreased blood perfusion to body tissues. As such, whole body toxicity precludes systemic delivery of medicaments as a way to achieve therapeutic concentrations in the globe's interior. If systemic aminoglycoside antibiotics are administered to treat eye infections, renal toxicity and ototoxicity are genuine concerns and will limit the amount of drug that can be systemically administered.

Retrobulbar injection, a somewhat targeted, non-systemic delivery method has been used since the 1920's. In this method, a bolus of drug is injected into the eye socket behind the eye. The drug then diffuses by passive diffusion into and through the tissues it contacts, including the sclera. Other methods have developed through the years, including sub-Tenon's capsule, peribulbar, and subconjunctival injections, all of which involve invasive delivery methods for injecting large amounts of a drug into a periocular space. Through injection to areas surrounding the globe, these methods achieve a high local concentration of the drug, allowing for trans-scleral drug delivery to the posterior chamber by passive, Fickian-driven diffusion. The injections, however, carry significant risks, including pain, risk of infection, tissue scarring, retrobulbar hemorrhage, ecchymosis, elevated intraocular pressure, accidental perforation of the globe, and proptosis. Secondly, there is no guarantee of achieving therapeutic drug concentrations in the vitreous following peribulbar injections. This is often the case with peribulbar antibiotic administration. Lastly, because diffusion does not occur unidirectionally into the globe, adverse reactions as a result of the systemic toxicity of the drug can still occur.

Another method for introducing medicament into the eye is by direct injection into the vitreous. Intravitreal injections have been used to deliver antibacterial and antifungal agents for treating bacterial and fungal endophthalmitis, to deliver antivirals for treating viral retinitis, to deliver steroids for treating uveitis, and to deliver antiangiogenics for treating age-related macular degeneration (ARMD) and diabetic retinopathy. The half-life of most compounds in the vitreous, however, is relatively short, usually on the scale of just a few hours. Therefore, intravitreal injections must sometimes be repeated, sometimes multiple times weekly. Each injection can cause pain, discomfort, intraocular pressure increases, intraocular bleeding, increased chances for infection, and a significant possibility of retinal detachment.

Yet another method for intraocular drug delivery is to implant drug-containing matrices. Such sustained-release drug delivery devices may be bioerodible or non-erodible. They commonly must be, however, surgically implanted into the interior of the globe to be effective. Once the drug payload is exhausted, a new matrix may be inserted to replace the old, or the old device left in place and a new matrix inserted nearby. Thus, such devices carry with them significant risks. Beside risks associated with surgery, the delivery device may cause pain and discomfort, induce intraocular bleeding, increase intraocular pressure, bring about infection, and contribute to retinal detachment. When ocular drug toxicity is observed, such as increased intraocular pressure or cataractogenesis during implantation therapy, the toxicity has to be managed or the device must be surgically removed.

In short, the present invention overcomes the disadvantages associated with known ocular drug delivery technologies. Because ocular tissue is highly permeable, the invention is particularly suited for precise and controlled drug delivery to the eye. Generally, there is no need to permeabilize eye tissue, though permeabilizing means may be used when the permselective material has a low resistance. As ocular tissue tends to be more sensitive than other tissue, it is preferred that iontophoresis be carried out with a single electrode in contact with ocular tissue. Any additional electrodes used in ocular iontophoresis are preferably spaced sufficiently far apart from the electrode in contact with ocular tissue such that the additional electrodes cannot simultaneously contact ocular tissue.

With a high degree of control over iontophoretic flux, the invention also may be advantageously utilized in transdermal drug delivery, or in transbuccal or other transmucosal routes of drug delivery. In general, iontophoretic methods are advantageous over oral delivery methods because gastrointestinal drug degradation, hepatic first-pass effects, and stomach upset and ulcerogenic effects are reduced if not eliminated. Although passive transdermal and transmucosal delivery systems do not require electrical power and are generally less costly than iontophoretic delivery devices, relatively few drugs have been found to be suitable for passive delivery through dermal and mucosal tissues. In contrast, electrically assisted transdermal or transmucosal iontophoretic delivery techniques have the ability to deliver, at sufficiently high fluxes to achieve therapeutically effective rates, many drugs, including drugs having high molecular weights such as polypeptides and proteins, which cannot be delivered at therapeutically effective rates by passive transdermal delivery systems.

Even when the drug to be delivered can be delivered transdermally by either active or passive techniques, electrically assisted iontophoretic delivery techniques are advantageous. For example, the inventive techniques allow pharmacologically effective transdermal drug delivery rates to be reached in a shorter amount of time. That is, the invention allows pharmacologically effective transdermal delivery rates to be achieved within several minutes of start-up, whereas passive transdermal delivery systems typically exhibit long onset times, on the order of an hour or more. In addition, the iontophoretic techniques described herein provide a greater degree of control over the amount and rate of drug delivered. Furthermore, iontophoresis allows for programmed drug delivery in a predetermined regimen, e.g., as bolus dose or “on demand” in applications such as the delivery of narcotic analgesics for treatment of pain.

Variations of the invention, not explicitly disclosed herein, will be apparent to those of ordinary skill in the art. For example, U.S. patent application Ser. No. 10/138,723, entitled “Device and Method for Monitoring and Controlling electrical Resistance at a Tissue Site Undergoing Electrophoresis,” filed May 3, 2002, inventors Miller, Higuchi, Li, and Hastings, describes an iontophoretic device that monitors the electrical resistance of the localized region undergoing electrophoresis by measuring any voltage difference between a reference electrode and at least one electrophoretic electrode. Thus, the iontophoretic system described herein may also include a third electrode adapted to contact the individual's body and be spaced apart from the first and second electrodes, and a means for determining the voltage between the third electrode and at least one of the first and second electrodes. In addition, multiple sources of electrical current may be provided for different purposes. Such sources of electrical current may generate signals of the same or a different type. For example, a DC source may be used to effect direct iontophoretic delivery and an AC source may be used to effect electroporation.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All patents, patent applications, patent publications and non-patent literature references mentioned herein are incorporated by reference in their entireties.

### Experimental

The practice of the present invention will employ, unless otherwise indicated, conventional techniques of pharmaceutical formulation, and the like, which are within the skill of the art. Such techniques are explained fully in the literature. Preparations of various types of pharmaceutical formulations are described, for example, in Remington: The Science and Practice of Pharmacy, Nineteenth Edition. (1995) and Ansel et al., Pharmaceutical Dosage Forms and Drug Delivery Systems, 6^{th} Ed. (Media, Pa.: Williams & Wilkins, 1995).

The following examples are put forth so as to provide those of ordinary skill in the art with a complete disclosure and description of how to make and use the compounds of the invention, and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. and pressure is at or near atmospheric. All components were obtained commercially unless otherwise indicated.

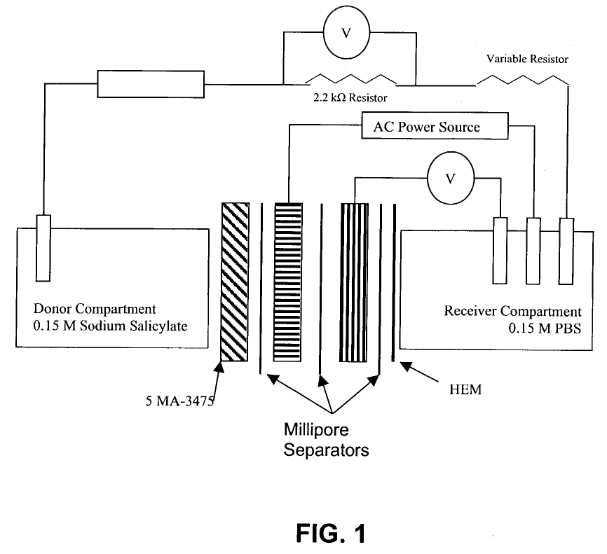

FIG. 1 schematically depicts the setup employed to carry out human epidermal membrane (HEM) iontophoretic experiments described below. Scleral experiments were conducted using a simple side-by-side diffusion apparatus known to one of ordinary skill in the art. Conductive silver paint was purchased from Ladd Research Technologies (Williston, Vt.) and silver foil from EM-Science (Gibbstown, N.J.). Silver chloride powder, phosphate buffered saline (PBS, pH 7.4) tablets were purchased from Sigma (St. Louis, Mo.). ¹⁴C-sodium salicylate was purchased from ARC (St. Louis, Mo.) and non-radiolabeled sodium salicylate from Sigma. Ultimate Gold® scintillation cocktail was purchased from Packard (Meriden, Conn.) and liquid scintillation counting was performed by a Packard TriCarb Model 1900 TR liquid scintillation analyzer. A custom built AC waveform generator power supply (EM-Tech Electronics, Lindon Utah) was used as the permeabilzation power supply for skin studies. The DC driving force for the skin studies was a 9 V battery (Duracell) combined with a fixed resistor. For scleral studies, there was no permeabilization source needed and all iontophoresis was conducted using a Phoresor PM-800 (Iomed, Inc., Salt Lake City, Utah). Human epidermal membrane was obtained from licensed sources and experiments were conducted under local IRB approval. Scleral tissue was obtained from freshly euthanized rabbit cadavers under local IACUC approval. All water was >18 MΩ prepared by the Milli-Q process. Ionac® MA-3475 (Sybron Chemical, Inc., Birmingham, N.J.) was pre-loaded with sodium salicylate by vigorously stirring the membranes in a 0.15 M sodium salicylate solution spiked with the radiolabel. Loading was considered complete when the DPM counts in the loading solution varied by less than 10% over a 12 hour period. The MA-3475 membranes were assembled in a group of five membranes and placed in the diffusion cell setup. The resistance of the MA-3475 stack was 500 Ω.

### EXAMPLE 1

Permeabilized skin transport experiments were conducted using a side-by-side type diffusion cell with an open diffusional area of 0.85 Cm². The cells were separated by a piece of dermatomed, heat-separated human epidermal membrane with the stratum corneum facing the donor compartment. Each side of the diffusion cell had a 2 ml volume and was stirred at 350 rpm with a magnetic stir bar.

The DC iontophoresis driving electrodes were prepared by dipping a silver foil strip into a 1:1 (w/w) mixture of conductive silver paint and finely ground silver chloride. After dipping, the electrodes were hung and allowed to cure at room temperature overnight. The AC permeabilizing electrodes were made by mechanically drilling holes into a piece of silver foil and electro-coating the perforated silver foil with AgCl by submersion in a saturated KCl solution and passing a 1 mA current for 10 minutes.

The receiver compartment was filled with 0.15 M PBS. In each experiment, the donor compartment contained 0.15 M sodium salicylate in water spiked with 25 nCi/ml ¹⁴C-sodium salicylate.

A 1000 Hz AC potential was applied to decrease and maintain the skin resistance to 2 and 0.5 kΩ and a 9 V battery was connected to a fixed resistor in series to yield a direct current of 0.5 mA. Every 30 minutes, the entire volume of the receiver solution was removed, mixed with scintillation cocktail, and analyzed by liquid scintillation counting. All experiments were conducted at least in duplicate.

The amount of ¹⁴C-salicylate transported across the membrane was plotted as a function of time. Permeabilities were determined from the following equations:

$$J = \frac{1}{A} \frac{ⅆ Q}{ⅆ t} \qquad ( 8 )$$

and

P=J/C_{D} (9)

where J is the flux, Q is the amount of solute transported across the membrane, A is the area of the exposed membrane, t is time, P is the permeability, and C_{D} is the concentration of the solute in the donor solution.

Permeabilities were plotted as a function of time and the slope of the best-fit line to the steady state portion of the curve was determined using regression analysis. All statistical analysis was accomplished using the statistical analysis package bundled with Microsoft™ Excel. Experimental results are presented in the Table 1.

TABLE 1

| DC Current | | | Transference |
|---|---|---|---|
| (mA) | PSM | Salicylate Flux (μg/min) | number |
| 0.5 | None | DC | 0.13 ± 0.01 |
| 0.5 | + | DC | 0.18 ± 0.03 |
| 0.5 | None | AC + DC, 0.5 kΩ | 0.12 ± 0.01 |
| 0.5 | + | AC + DC, 0.5 kΩ | 0.32 ± 0.03 |
| 0.5 | None | AC + DC, 2 kΩ | 0.12 ± 0.01 |
| 0.5 | + | AC + DC, 2 kΩ | 0.16 ± 0.03 |

Table 1 shows that when purely DC iontophoresis is conducted across HEM, the accentuation in transport provided by the permselective material is only minimal. Without the permselective material, 13% of the current is carried by the sodium salicylate whereas 18% of the current is carried by the drug when the permselective material is present, representing a gain of only about 40%. Similarly, during the AC permeabilization experiment with a target human epidermal membrane resistance of 2 kΩ, 12% of the current was carried by the drug in the absence of an permselective membrane and only 16% in its presence, an increase of about 30%. When the target resistance of the human epidermal membrane was lowered to 0.5 kΩ, however, the electrical transference efficiency increased from 12% to 32%, almost 300%.

This example demonstrates utility of the permselective membrane in increasing ion flux through a biological membrane, the human skin. This example also demonstrates the need for the membrane to be highly permeabilized. When direct current DC was used to conduct iontophoresis, it was unable to permeabilize the human epidermal membrane to a sufficient degree for the permselective membrane to dominate drug transport through the system. This inability to achieve sufficient permeabilization was exhibited despite the use of 0.5 mA DC (0.6 mA/cm²), a value which exceeds generally recognized as safe limits. Likewise, when alternating current AC was used to permeabilize the skin to 2 kΩ, the enhancement afforded by the permselective membrane was negligible. However, when alternating current was used to permeabilize the skin to a very low resistance, 0.5 kΩ, the enhancement in electrical transference jumped by an order of magnitude, further demonstrating the need to achieve a comparable, or higher, level of permeability in the tissue that of the permselective membrane (500 Ω).

This study demonstrates that the current invention can be used to enhance drug transport through biomembranes. In addition, this study contrasts the important difference with the prior art: the need for a highly permeabilized tissue. Unpermeabilized or moderately permeabilized tissues, or tissues permeabilized with pure DC do not demonstrate utility with permselective membranes.

### EXAMPLE 2

Scleral transport experiments were conducted using a side-by-side type diffusion cell with an open diffusional area of 0.2 cm². The cells were separated by a piece of excised scleral tissue from adult rabbits. The diffusion cells were assembled with the conjunctival side facing the donor compartment. Each side of the diffusion cell had a 2 ml volume and was stirred at 350 rpm with a magnetic stir bar.

The DC iontophoresis driving electrodes were prepared by dipping a silver foil strip into a 1:1 (w/w) mixture of conductive silver paint and finely ground silver chloride. After dipping, the electrodes were hung and allowed to cure at room temperature overnight. Because of the inherently high permeability of the sclera, it was not necessary to use an AC permeabilizing electrodes for this study.

The receiver compartment was filled with 0.15 M PBS. In each experiment, the donor compartment contained 0.15 M sodium salicylate in water spiked with 25 nCi/ml ¹⁴C-sodium salicylate.

Direct current DC was applied at currents of 0.5, 1, and 2 mA with a Phoresor. Every 30 minutes, the entire volume of the receiver solution was removed, mixed with scintillation cocktail, and analyzed by liquid scintillation counting. All experiments were conducted in triplicate.

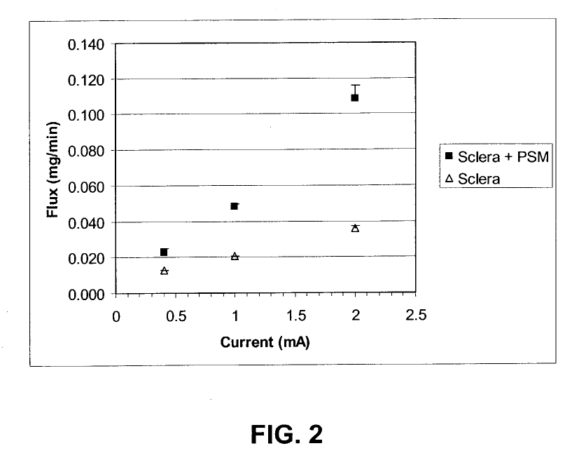

The amount of ¹⁴C-salicylate transported across the membrane was plotted as a function of time. Permeabilities were determined from the equations (8) and (9) and plotted and analyzed as in Example 1. Experimental results are presented in Table 2 and graphically depicted in FIG. 2.

TABLE 2

| DC Current | | Resistance | Salicylate Flux | Transference |
|---|---|---|---|---|
| (mA) | PSM | (kΩ) | (μg/min) | number |
| 0.4 | None | 1.0 | 13 ± 0.1 | 0.385 ± 0.007 |
| 0.4 | + | 1.9 | 21 ± 0.3 | 0.675 ± 0.073 |
| 1.0 | None | 1.5 | 21 ± 0.2 | 0.235 ± 0.007 |
| 1.0 | + | 2.5 | 48 ± 1.0 | 0.563 ± 0.013 |
| 2.0 | None | 2.3 | 36 ± 2.0 | 0.210 ± 0.014 |
| 2.0 | + | 2.3 | 109 ± 7.0 | 0.650 ± 0.048 |

Table 2 and FIG. 2 show that salicylate flux is proportional to DC current level across the sclera. These figures also show that the permselective membrane enhances the salicylate flux across the sclera two- to three-fold, depending on the current level. This table depicts the transference number as a function of current level for both the biomembrane and the biomembrane plus the permselective material. As shown, and expected for a membrane with relatively high inherent permeability, the transference number is independent of current level for a given condition. The presence of the permselective membrane increased the transference number between two- and three-fold.

This study demonstrates the utility of the present invention towards increasing the flux of a model permeant across the sclera. With this system, approximately 65% of the current is carried by the drug, leaving only 35% of the electrical current carried by competing ions. This example demonstrates the invention using a permeable biomembrane combined with a permselective membrane. However, this example should not be considered limiting. With further optimization, it is expected that electrical transferences in excess of 95% will be achieved.

We claim:

1. An iontophoretic method for selectively transporting a compound of interest through a localized region of an individual's body tissue, wherein the localized region exhibits an electrical resistance less than 100 kΩ-cm² the method comprising:
(a) placing a permselective material in ion-conducting relation to the localized region, wherein the permselective material is capable of hindering iontophoretic transport of a competing ion that reduces transference efficiency of the compound of interest; and
(b) applying an electrical current through the permselective material to the localized region, thereby transporting the compound of interest iontophoretically through the localized region while hindering iontophoretic transport of the competing ion.

2. The method of claim 1, wherein the permselective material has an electrical resistance greater than the electrical resistance of the localized region.

3. The method of claim 1, wherein the localized region exhibits an electrical resistance no greater than about 10% of the electrical resistance of unpermeabilized skin tissue.

4. The method of claim 3, wherein the localized region exhibits an electrical resistance no greater than about 1% of the electrical resistance of unpermeabilized skin tissue.

5. The method of claim 1, wherein the individual is a mammal.

6. The method of claim 5, wherein the individual is human.

7. The method of claim 1, wherein the body tissue is mucosal tissue.

8. The method of claim 7, wherein the mucosal tissue is oral tissue.

9. The method of claim 8, wherein the oral tissue is buccal tissue.

10. The method of claim 1, wherein the body tissue is ocular tissue.

11. The method of claim 10, wherein the ocular tissue is scleral tissue.

12. The method of claim 10, wherein the ocular tissue is conjunctival tissue.

13. The method of claim 1, wherein the localized region is permeabilized before step (b) so as to exhibit an electrical resistance that corresponds to a tissue permeability that exceeds the permeability of the individual's unpermeabilized skin tissue.

14. The method of claim 13, wherein the electrical resistance of the localized region is less than 10 kΩ-cm².

15. The method of claim 14, wherein the electrical resistance of the localized region is less than 5 kΩ-cm².

16. The method of claim 15, wherein the electrical resistance of the localized region is less than 2 kΩ-cm².

17. The method of claim 13, wherein permeabilization reduces the electrical resistance of the localized region by at least about 90%.

18. The method of claim 17, wherein permeabilization reduces the electrical resistance of the localized region by at least about 99%.

19. The method of claim 13, wherein the tissue is permeabilized through use of a chemical permeation enhancer, electroporation current, ultrasound, photons, a piercing member, and/or combinations thereof.

20. The method of claim 13, wherein the body tissue is permeabilized mucosal tissue.

21. The method of claim 13, wherein the body tissue is permeabilized skin tissue.

22. The method of claim 1, wherein the electrical resistance of the permselective material is at least two times the electrical resistance of the localized region.

23. The method of claim 22, wherein the electrical resistance of the permselective material is at least five times the electrical resistance of the localized region.

24. The method of claim 23, wherein the electrical resistance of the permselective material is at least ten times the electrical resistance of the localized region.

25. The method of claim 24, wherein the electrical resistance of the permselective material is at least 20 times the electrical resistance of the localized region.

26. The method of claim 1, wherein the permselective material is provided in the form of a membrane.

27. The method of claim 1, wherein the permselective material is capable of hindering iontophoretic transport of a competing counter-ion that possesses a charge opposite to the charge of the compound of interest when ionized.

28. The method of claim 27, wherein the competing ion is negatively charged.

29. The method of claim 27, wherein the competing ion is positively charged.

30. The method of claim 1, wherein the electrical current comprises an alternating current.

31. The method of claim 30, wherein the electrical current further comprises a superimposed direct current.

32. The method of claim 1, wherein the electrical current comprises a direct current.

33. The method of claim 1, wherein the compound of interest is delivered into the body tissue.

34. The method of claim 1, wherein the compound of interest is delivered through the body tissue.

35. The method of claim 1, wherein the compound of interest is extracted from the body tissue.

36. The method of claim 35, wherein the extracted compound is endogenous to the body tissue.

37. The method of claim 36, wherein the extracted compound is glucose.

38. The method of claim 36, wherein the extracted compound is phenylalanine.

39. The method of claim 1, wherein the compound of interest is a pharmacologically active agent.

40. An iontophoretic electrode assembly for selectively transporting a compound of interest through a localized region of an individual's body tissue, wherein the localized region exhibits an electrical resistance that corresponds to a tissue permeability that exceeds the permeability of the individual's unpermeabilized skin tissue, the electrode assembly comprising:
an electrode adapted for electrical connection to a current source; and
a permselective material in ion-conducting relation to the electrode and having a surface adapted for contact with the localized region,
wherein the permselective material has an electrical resistance greater than the electrical resistance of the localized region and is capable of hindering iontophoretic transport of a competing ion that reduces transference efficiency of the compound of interest when the material is in contact with the localized region.

41. The electrode assembly of claim 40, wherein the permselective material is a membrane.

42. The electrode assembly of claim 40, wherein the membrane has a surface sized and/or shaped for direct contact with the localized region.

43. The electrode assembly of claim 42, wherein the body tissue is buccal tissue.

44. The electrode assembly of claim 42, wherein the body tissue is skin tissue.

45. The electrode assembly of claim 42, wherein the body tissue is ocular tissue.

46. The electrode assembly of claim 45, wherein the ocular tissue is scleral tissue.

47. The electrode assembly of claim 45, wherein the ocular tissue is conjunctival tissue.

48. The electrode assembly of claim 40, wherein the membrane is capable of hindering iontophoretic transport of a competing counter-ion that possesses a charge opposite to the charge of the compound of interest when ionized.

49. The electrode assembly of claim 40, further comprising a reservoir for containing the compound of interest, wherein the reservoir is in electrical contact with the electrode and the permselective material.

50. The electrode assembly of claim 49, wherein the reservoir is interposed between the electrode and the permselective material.

51. The electrode assembly of claim 50, further comprising a means for isolating the reservoir so as to prevent a redox product from entering the reservoir.

52. The electrode assembly of claim 42, wherein the means for isolating the reservoir is comprised of an agent that precipitates, neutralizes, repels, and/or binds to the redox product so as to prevent the product from entering the reservoir.

53. The electrode assembly of claim 51, wherein the means for isolating the reservoir is comprised of an additional permselective material that selects for the charge of the compound of interest when ionized.

54. The electrode assembly of claim 53, wherein the additional permselective material is a membrane.

55. The electrode assembly of claim 50, wherein the compound of interest is contained within the reservoir.

56. The electrode assembly of claim 40, wherein the compound of interest is contained within the permselective material.

57. The electrode assembly of claim 40, wherein the compound of interest is a pharmacologically active agent.

58. The electrode assembly of claim 40, further comprising a means for permeabilizing the localized region such that the region exhibits an electrical resistance no greater than about 20% of the electrical resistance of unpermeabilized tissue.

59. The electrode assembly of claim 58, wherein the means for permeabilizing the localized region permeabilizes the localized region such that the region exhibits an electrical resistance no greater than about 20% of the electrical resistance of unpermeabilized mucosal tissue.

60. The electrode assembly of claim 58, wherein the means for permeabilizing the localized region permeabilizes the localized region such that the region exhibits an electrical resistance no greater than about 20% of the electrical resistance of unpermeabilized skin tissue.

61. The electrode assembly of claim 58, wherein the means for permeabilizing the localized region comprises a chemical permeation enhancer.

62. The electrode assembly of claim 61, wherein the means for permeablizing the localized region further comprises an applicator that applies the chemical permeation enhancer to the tissue prior to iontophoresis.

63. The electrode assembly of claim 61, wherein the chemical permeation enhancer is in ion-conducting relation to the permselective material.

64. The electrode assembly of claim 61, wherein the permeation enhancer is contained in the permselective material.

65. The electrode assembly of claim 58, wherein the means for permeabilizing the localized region comprises a permeabilizing current applicator spaced apart from the electrode.

66. The electrode assembly of claim 65, wherein the permselective material is interposed between the electrode and the permeabilizing current applicator.

67. An iontophoretic system for selectively transporting a compound of interest through a localized region of an individual's body tissue, comprising:
a permselective material capable of selectively hindering iontophoretic transport of a competing ion when the material is in contact with the localized region, wherein transport of the competing ion reduces transference efficiency of the compound of interest;
a first electrode adapted to be placed in ion-conducting relation through the permselective material to the localized region to allow iontophoretic transport of the compound therethrough;
a second electrode adapted to contact the individual's body and spaced apart from the first electrode; and
a current source electrically connected to the first and second electrodes, for applying an electrical current to the localized region of body tissue to transport the compound of interest iontophoretically through the localized region; and
a means for permeabilizing the localized region such that the permselective material has an electrical resistance greater than the electrical resistance of the localized region after permeabilization by the means for permeabilizing the localized region.

68. The system of claim 67, wherein the second electrode is spaced sufficiently far apart from the first electrode such that the electrodes cannot simultaneously contact the body tissue of the localized region.

69. The system of claim 68, wherein the first electrode is adapted to contact ocular tissue.

70. The system of claim 69, further comprising a third electrode adapted to contact the individual's body and spaced apart from the first and second electrodes, and a means for determining the voltage between the third electrode and at least one of the first and second electrodes.

71. An iontophoretic system for selectively transporting a compound of interest through a localized region of an individual's body tissue, comprising:
a permselective material capable of hindering iontophoretic transport of a competing ion when the material is in contact with the localized region, wherein transport of the competing ion reduces transference efficiency of the compound of interest;
a first electrode adapted to be placed in ion-conducting relation through the permselective material with the localized region to allow iontophoretic transport of the compound therethrough;
a second electrode adapted to contact the individual's body tissue and spaced apart from the first electrode; and
an alternating current source electrically connected to the first and second electrodes, for applying an alternating current to the localized region to transport the compound of interest iontophoretically through the localized region.

72. The system of claim 71, wherein the alternating current source applies sufficient current to lower the electrical resistance of the localized region to a level less than the electrical resistance of the permselective material.

73. The system of claim 72, wherein the first electrode is a perforated electrode.

74. The system of claim 73, further comprising a reservoir containing the compound of interest, wherein the first electrode is interposed between the reservoir and the permselective material.

75. The system of claim 71, further comprising a third electrode adapted to be placed between the permselective membrane and the localized region.

76. The system of claim 71, further comprising a direct current source.

77. The system of claim 76, wherein the direct current source is connected to at least one of the first and second electrodes.

78. The system of claim 71, where the permselective material comprises a polyelectrolyte capable of transferring an electrical current interdispersed in an insulating material.
